(19) United States
(12) Patent Application Publication
(10) Pub. No.: US 2008/0168586 A1
(43) Pub. Date: Jul. 10, 2008

(54) Brassica Plant Comprising a Mutant Fatty Acid Desaturase

(75) Inventors: Benjamin Laga, Wingene (BE); Dirk Decherf, Jabbeke (BE); Jozef Seurinck, Nazareth (BE)

Correspondence Address: CONNOLLY BOVE LODGE & HUTZ, LLP, P O BOX 2207, WILMINGTON, DE, 19899, US

(73) Assignee: BAYER BIOSCIENCE N.V., GENT (BE)

(21) Appl. No.: 11/883,235

(22) PCT Filed: Jan. 26, 2006
(86) PCT No.: PCT/EP2006/001445

§ 371 Date: Mar. 19, 2008

Related U.S. Application Data
(60) Provisional application No. 60/647,902, filed on Jan. 28, 2005.

(51) Int. Cl. A01H 5/00; C12N 15/52; C12N 5/10; A01H 1/02; C07H 21/04; C12Q 1/68; C11B 1/00; C07K 14/00

(52) U.S. Cl. 800/306; 536/23.2; 435/419; 530/350; 554/8; 800/260; 435/6; 536/24.33

(57) ABSTRACT

The present invention relates to plants, particularly Brassica plants, and parts of plants having genes and expressing enzymes that affect fatty acid composition. More particularly, this invention relates to nucleic acids encoding a delta-12 fatty acid desaturase protein that affect fatty acid composition in plants. Furthermore, the present invention relates to methods for the manufacture of such plants.

### FIELD OF THE INVENTION

This invention relates to plants and parts of plants having genes and expressing enzymes that affect fatty acid composition.

This invention also relates to a fatty acid desaturases and nucleic acids encoding desaturase proteins. More particularly, this invention relates to nucleic acids encoding a delta-12 fatty acid desaturase protein that affect fatty acid composition in plants.

### BACKGROUND OF THE INVENTION

Vegetable oils are increasingly important economically because they are widely used in human and animal diets and in many industrial applications. However, the fatty acid compositions of these oils are often not optimal for many of these uses. Because specialty oils with particular fatty acid composition are needed for both nutritional and industrial purposes, there is considerable interest in modifying oil composition by plant breeding an d/or by new molecular tools of plant biotechnology.

Brassica species like Brassica napus (B. napus) and Brassica rapa (B. rapa) constitute the third most important source of vegetable oil in the world. In Canada, plant scientists focused their efforts on creating so-called “double-low” varieties which were low in erucic acid in the seed oil and low in glucosinolates in the solid meal remaining after oil extraction (i.e., an erucic acid content of less than 2.0 percent by weight based upon the total fatty acid content, and a glucosinolate content of less than 30 micromoles per gram of the oil-free meal). These higher quality forms of rape developed in Canada are known as canola.

Among the fatty acids, the polyunsaturated fatty acids linoleate (C18:2) and α-linolenate (C18:3) are essential fatty acids for human nutrition. They are synthesized by plants but not by most other higher eukaryotes.

In Angiosperm as a whole, the vast majority of polyunsaturated lipid synthesis passes through a single enzyme, the delta-12 desaturase (also called oleate desaturase or FAD2 desaturase) of the endoplasmic reticulum. Furthermore, it is responsible for more than 90% of the polyunsaturated fatty acid synthesis in non photosynthetic tissues such as developing seed of oil crops including canola, in which fatty acids are stored as triacylglycerol oils.

The FAD2 desaturase is involved in enzymatic conversion of oleic acid to linoleic acid. A microsomal FAD2 desaturase has been cloned and characterized using T-DNA tagging (Okuley et al., Plant cell 6: 147-158 (1994)).

The nucleotide sequences of higher plant genes encoding microsomal FAD2 desaturase is described in WO 94/11516. The WO 97/21340, WO98156239, U.S. Pat. No. 5,850,026, U.S. Pat. No. 6,063,947, U.S. Pat. No. 6,441,278 and EP 945 514 describe FAD2 desaturase, Brassica seeds and plants having mutant sequences which confer altered fatty acid profiles on seed oil.

### SUMMARY OF THE INVENTION

The present invention relates to Brassica seeds, plants and parts of plants comprising a new mutation in the FAD2 gene. The present invention also relates to the isolation and characterization of a new Isolated nucleic acid sequence encoding a mutant FAD2 protein conferring an altered fatty acid composition in seed oil when present in the plant, e.g., a high oleic acid content and a low linoleic acid content.

Methods are provided to obtain plants containing such mutant FAD2 allele (fad2) and to assess the presence of such mutant FAD2 allele (fad2) using PCR primers.

Marker assisted plant breeding programs are provided by the invention, wherein the mutant FAD2 allele (fad2) of the invention may be identified in plant lines subjected to selective breeding.

Methods are also provided for using the plants of the invention, including selected plants and transgenic plants, to obtain plant products. As used herein, “plant product” includes anything derived from a plant of the invention, including plant parts such as seeds, meals, fats or oils.

### BRIEF DESCRIPTION OF THE SEQUENCE LISTING

SEQ ID No. 1: DNA sequence of the wild type Brassica rapa FAD2A allele

SEQ ID No. 2: DNA sequence of a wild type Brassica napus FAD2A allele

SEQ ID No. 3: DNA sequence comprising part of the mutant Brassica napus FAD2 allele (fad2)

SEQ ID No. 4: deduced amino acid sequence of SEQ ID No. 3

SEQ ID No. 5: deduced amino acid sequence of SEQ ID No. 2

SEQ ID No. 6: deduced amino acid sequence of SEQ ID No. 1

SEQ ID No 7: PCR primer OSR144.

SEQ ID No. 8: PCR primer OSR145

SEQ ID No. 9: PCR primer OSR146

SEQ ID No. 10: PCR primer OSR147

SEQ ID No. 11: PCR primer OSR001

SEQ ID No. 12: PCR primer OSR002

### BRIEF DESCRIPTION OF THE DRAWING

FIG. 1 represents the alignment of the fad2 nucleic acid of the invention with FAD2 nucleic sequences of the public database, “FAD2A-WT-Brapa” represents the wild type FAD2 gene of the A genome of B. rapa (SEQ ID No. 1), “FAD2A-WT-Bnapus” represents the wild type FAD2 gene of A genome of B. napus (SEQ ID No. 2) and “FAD2-Mutant” represents the fad2 nucleic acid sequence of the invention (SEQ ID No. 3).

FIG. 2 represents the alignment of the deduced amino acid sequence of the FAD2 polypeptide originating from B. rapa (SEQ ID No. 6), B. napus (SEQ ID No. 5) and the mutant FAD2 protein of the invention (SEQ ID No. 4).

FIG. 3 represents the correlation between the presence of the fad2 allele from HOWOSR in homozygous and heterozygous state and the level of oleic acid in seed oil in the greenhouse.

FIG. 4 represents the correlation between the presence of the fad2 allele from HOWOSR and the level of oleic acid in seed oil in the field.

FIG. 5 represents the correlation between the presence of the fad2 allele from HOWOSR and the level of oleic acid in seed oil of progeny plants of crosses involving plants having the fad2 allele and plants having the fad3a and fad3c alleles.

FIG. 6 represents the correlation between the presence of the fad3a and fad3c alleles from B3119 Stellar and the level of linolenic acid in seed oil of progeny plants of crosses involving plants having the fad2 allele and plants having the fad3a and fad3c alleles.

### DETAILED DESCRIPTION OF THE EMBODIMENTS

In one aspect, the invention provides an isolated nucleic acid, encoding a mutant FAD2 desaturase, comprising a nucleotide deletion. In a specific aspect of the invention, such an isolated nucleic acid comprises the nucleotide sequence of SEQ ID No. 3.

By “isolated” is meant that the isolated substance has been substantially separated or purified from other biological components. “Isolated” also includes the substances purified by standard purification methods, as well as substances prepared by recombinant expression in a host, as well as chemically synthesized substances.

In another aspect, the invention deals with a mutant FAD2 polypeptide encoded by an nucleic acid which comprises a nucleotide deletion, said FAD2 polypeptide being non functional. In a specific aspect of the invention, the mutant FAD2 polypeptide comprises an amino acid sequence represented by SEQ ID No. 4.

“Mutant FAD2 desaturase” according to the invention refers to a polypeptide encoded by a nucleic acid comprising a mutation, and more particularly said mutant FAD2 desaturase comprises SEQ ID No. 4.

“Mutant FAD2 nucleic acid (fad2)” according to the invention refers to a nucleic acid comprising a deletion, and more particularly it refers to an isolated nucleic acid comprising SEQ ID No. 3.

“Mutant FAD2 allele (or fad2 allele)”: shall be understood according to the present invention as the particular form of the FAD2 gene that comprises a deletion and more particularly to a FAD2 gene comprising SEQ ID No. 3.

The isolated nucleic acid of the invention comprises a mutation within the coding sequence of the FAD2 desaturase gene. The nucleic acid fragment of the invention may be in the form of a gene, a RNA, a cDNA. The DNA could be in single- or double-stranded form, it can be either the coding or non-coding strand. The RNA may be in the form of a mRNA or the corresponding antisense RNA or part of it.

In one aspect of the invention, the mutation is a frameshift mutation that results in nonsense translation and premature stop. Such a mutation renders the resulting FAD2 desaturase non-functional in plants, relative to the function of the gene product encoded by the wild type sequence. The non-functionality of the FAD2 desaturase protein leads to a decreased level of linoleic acid and an increased level of oleic acid in seed oil of plants expressing the mutant sequence, compared to the corresponding levels in seed oil of plants expressing the non-mutant sequence.

Another aspect of the invention refers to plant cells comprising the mutant FAD2 nucleic acid and more particularly comprising SEQ ID No. 3 or expressing a mutant FAD2 polypeptide and more particularly expressing a polypeptide comprising SEQ ID No. 4.

In a diploid species there are two alleles present at a given locus, although more than two alleles for the locus may exist in the population. If the two alleles at a corresponding locus of homologous chromosomes are the same, one refers to the locus as being homozygous. For example double haploid (DH) plants, which are generated by chromosome doubling, are homozygous at all loci. If the two alleles at a corresponding locus of homologous chromosomes are not the same, one refers to the locus as being heterozygous. Brassica napus (B. napus, 2n=38, genome MCC) is an amphidiploid species, which originated from a spontaneous hybridization of Brassica rapa L. (syn. B. campestris; 2n=20, AA) and Brassica oleracea L. (2n=18, CC). B. napus contains the complete chromosome sets of these two diploid genomes.

Another aspect of the invention refers to Brassica plants and/or Brassica plant parts comprising such a mutant FAD2 allele (fad2) and more particularly comprising SEQ ID No. 3 or expressing a polypeptide comprising SEQ ID No. 4.

Such plants present an alteration of the fatty acid composition of the seed oil, e.g., an altered level of oleic acid (18:1) and linoleic acid (18:2). The Brassica plants of the present invention contain 59.9 to 75.6% of oleic acid and 9.3 to 14.3% of linoleic acid based on the total fatty acid content of the seed.

In a further embodiment of the invention, the mutant FAD2 nucleic acid (fad2) in an antisense form has been transferred to a Brassica plant by genetic transformation. The transformed plants or cells, comprising such mutant FAD2 nucleic acid (fad2) constitute another aspect of the invention.

In a further embodiment, the invention refers to a Brassica plant, with high oleic acid levels in its seed oil, comprising the mutant FAD2 allele, wherein said Brassica plant is selected from the group consisting of:
a Brassica plant containing a transgene integrated into its genome,
a Brassica plant that contains a level of aliphatic glucosinolates in dry, defatted seed meal of less than 30 micromol/g,
a Brassica plant the solid component of the seed contains less than 30 micromoles of any one or any mixture of 3-butenyl glucosinolate, 4-pentenyl glucosinolate, 3-hydroxy-3 butenyl glucosinolate, and 2-hydroxy-4-pentenyl glucosinolate per gram of air-dry, oil-free solid,
a Brassica plant that produces an oil containing less than 2% erucic acid of the total fatty acids in the oil,
a Brassica napus plant,
a B. napus spring oilseed rape plant,
a B. napus winter oilseed rape plant,

progeny of a Brassica plant containing said mutant FAD2 nucleic acid (fad2), wherein said progeny results from crosses between Brassica plants containing said mutant FAD2 nucleic acid (fad2) and a Brassica variety with low linolenic acid content in its seeds or a herbicide resistant Brassica variety.

In a more specific embodiment, the invention refers to a Brassica plant with high oleic acid content in its seeds comprising a mutant FAD2 nucleic acid (fad2) represented by SEQ ID No. 3 or mutant FAD2 polypeptide comprising SEQ ID No. 4, wherein said Brassica plant is selected from the group consisting of:
a Brassica plant containing a transgene integrated into its genome,
a Brassica plant that contains a level of aliphatic glucosinolates in dry, defatted seed meal of less than 30 micromol/g,
a Brassica plant the solid component of the seed contains less than 30 micromoles of any one or any mixture of 3-butenyl glucosinolate, 4-pentenyl glucosinolate, 3-hydroxy-3 butenyl glucosinolate, and 2-hydroxy-4-pentenyl glucosinolate per gram of air-dry, oil-free solid,
a Brassica plant that produces an oil containing less than 2% erucic acid of the total fatty acids in the oil,
a Brassica napus plant,
a B. napus spring oilseed rape plant,
a B. napus winter oilseed rape plant,
progeny of a Brassica plant containing said mutant FAD2 nucleic acid (fad2), wherein said progeny results from crosses between Brassica plants containing said mutant FAD2 nucleic acid (fad2) and a Brassica variety with low linolenic acid content in its seeds or a herbicide resistant Brassica variety.

The Brassica plants of the invention may additionally contain an endogenous gene or a transgene, which confers herbicide resistance, such as the bar or pat gene, which confers resistance to glufosinate ammonium (Liberty or Basta) (EP 0 242 236 and EP 0 242 246 incorporated by reference); or any modified EPSPS gene, such as the 2mEPSPS gene from maize (EP0 508 909 and EP 0 507 698 incorporated by reference), which confers resistance to glyphosate (RoundupReady).

In one embodiment of the invention, the plant can also contain a mutation in the delta-15 desaturase (FAD3 desaturase) conferring a high level of oleic acid and a very low level of alpha-linoleic acid in the seed oil. Such a mutation conferring low linolenic acid content is described in WO 98/56239 and WO 01/25453. Mutations in both FAD2 and FAD3 desaturase may be combined in a plant by making a genetic cross between FAD2 desaturase and FAD3 desaturase double mutant lines.

The plants of the present invention can be used to produce oilseed rape oil or an oilseed rape seed cake, to produce seed comprising a mutant FAD2 enzyme and more particularly to produce a crop of oilseed rape, comprising a mutant FAD2 enzyme.

Another aspect of the invention is a seed of a Brassica plant comprising the mutant FAD2 allele of the invention. This seed can be an inbred or a hybrid seed.

In a more specific aspect of the invention the seed is a hybrid B. napus seed, comprising the fad2 allele of the invention, wherein said hybrid B. napus seeds develop into plants, the solid component of the seeds contains less than 30 micromoles of any one or any mixture of 3-butenyl glucosinolate, 4-pentenyl glucosinolate, 3-hydroxy-3 butenyl glucosinolate, and 2-hydroxy-4-pentenyl glucosinolate per gram of air-dry, oil-free solid and produces an oil containing less than 2% erucic acid of the total fatty acids in the oil. In an even more specific aspect of the invention the hybrid seed is a hybrid B. napus seed, comprising SEQ ID No. 3, wherein said hybrid B. napus seeds develop into plants, the solid component of the seeds contains less than 30 micromoles of any one or any mixture of 3-butenyl glucosinolate, 4-pentenyl glucosinolate, 3-hydroxy-3 butenyl glucosinolate, and 2-hydroxy-4-pentenyl glucosinolate per gram of air-dry, oil-free solid and produces an oil containing less than 2% erucic acid of the total fatty acids in the oil.

The plants derived of such seeds are also part of the invention.

“Progeny” shall encompass the descendants of a particular plant or plant line, for example, seeds developed on a plant are descendants. Progeny of a plant include seeds formed on F1, F2, F3, S1, S2, S3 and subsequent generation plants or seeds formed on BC1, BC2, BC3, subsequent generation plants and DH plants.

Breeding procedures such as crossing, selfing, and backcrossing are well known in the art (see Allard R W (1960) Principles of Plant Breeding. John Wiley & Sons, New York, and Fehr W R (1987) Principles of Cultivar Development, Volume 1, Theory and Techniques, Collier Macmillan Publishers, London. ISBN 0-02-949920-8). The mutant FAD2 allele (fad2) of the invention can be transferred into other breeding lines or varieties either by using traditional breeding methods alone or by using additionally Marker Assisted Selection (MAS). The mutant FAD2 allele (fad2) can be transferred to the A-genome of B. juncea by interspecific crosses between B. napus and B. juncea (Roy (1984), Euphytica 295-303). The breeding program may involve crossing to generate an F1 (first filial generation), followed by several generations of selfing (generating F2, F3, etc.). The breeding program may also involve backcrossing (BC) steps, whereby the offspring is backcrossed to one of the parental lines (termed the recurrent parent).

Breeders select for agronomically important traits, such as high yield, high oil content, oil profile, flowering time, plant height, disease resistance, resistance to pod shattering, abiotic stress resistance, etc., and develop thereby elite breeding lines (lines with good agronomic characteristics). In addition, plants are bred to comply with quality standards, such as ‘canola’ quality (less than 30 μmoles per gram glucosinolates in oil-free meal and less than 2% by weight erucic acid in the oil, see, e.g., U.S. Pat. No. 6,303,849B1 for canola quality B. juncea).

The nucleic acid fragments of the invention can be used as markers or to develop markers in plant genetic mapping and plant breeding programs.

A “(molecular) marker” as used herein refers to a measurable, genetic characteristic with a fixed position in the genome, which is normally inherited in a Mendelian fashion, and which can be used for mapping of a trait of interest. The nature of the marker is dependent on the molecular analysis used and can be detected at the DNA, RNA or protein level. Genetic mapping can be performed using molecular markers such as, but not limited to, RFLP (restriction fragment length polymorphisms; Botstein et at. (1980), Am J Hum Genet. 32:314-331; Tanksley et al. (1989), Bio/Technology 7:257-263), RAPD (random amplified polymorphic DNA; Williams et al. (1990), NAR 18:6531-6535), AFLP (Amplified Fragment Length Polymorphism; Vos et al. (1995) NAR 23:4407-4414), SNPs or microsatellites (also termed SSR's; Tautz et al., (1989), NAR 17:6463-6471). Appropriate primers or probes are dictated by the mapping method used.

A molecular marker is said to be “linked” to a gene or locus, if the marker and the gene or locus have a greater association in inheritance than would be expected from independent assortment, i.e., the marker and the locus co-segregate in a segregating population and are located on the same chromosome. “Linkage” refers to the genetic distance of the marker to the gene or locus (or two loci or two markers to each other). Closer is the linkage, smaller is the likelihood of a recombination event between the marker and the gene or locus. Genetic distance (map distance) is calculated from recombination frequencies and is expressed in centiMorgans (cM) (Kosambi (1944), Ann. Eugenet. 12:172-175).

The present invention also deals with a kit for the detection of the mutant FAD2 allele (fad2), such a kit comprises PCR primers pairs for performing the mutant FAD2 (fad2) PCR Identification protocol. Said protocol allows the detection of the fad2 allele in DNA samples, a specific embodiment of this protocol is described in the following examples.

The kit for the detection of the mutant FAD2 allele (fad2) in DNA samples according to the present invention, comprises one or more PCR primer pairs, which are able to amplify a DNA marker linked to the mutant FAD2 gene (fad2), the wild type FAD2 gene and an endogeneous fragment of DNA.

Such a kit comprises PCR primer pairs selected from the following primer pairs OSR144 (SEQ ID No. 7)-OSR145 (SEQ ID No. 8), OSR146 (SEQ ID No. 9)-OSR147 (SEQ ID No. 10), and OSR001 (SEQ ID No. 11)-OSR002 (SEQ ID No. 12). The kit according to the present invention comprises said primer pairs which are able to amplify a DNA fragment of about 250, 101 and 394 bp, respectively. More particularly, the PCR primer pair which allows the amplication of a DNA marker linked to the mutant FAD2 gene (fad2) according to the present invention corresponds to primer pair OSR144 (SEQ ID No. 7)- and OSR145 (SEQ ID No. 8).

The kit may further comprise seeds or tissue, wherein DNA extracted from said seeds or tissue can be used as a positive or negative control.

According to another aspect of the invention, the PCR primers can be used for monitoring the introgression of mutant FAD2 nucleic acid (fad2) in Brassica oilseed rape plants or for PCR analysis of Brassica oilseed plants. More specifically the PCR primers OSR144 (SEQ ID No. 7) and-OSR145 (SEQ ID No. 8) are used for monitoring the introgression of the mutant FAD2 allele (fad2) in Brassica plants.

The present invention also encompasses a method for transferring the fad2 nucleic acid into another Brassica plant, comprising crossing the plant comprising the fad2 allele of the invention with another Brassica plant, collecting F1 hybrid seeds from said cross, selfing or crossing the F1 plants derived from said F1 seeds for one or more generations and screening plants derived from said selfing or crossing for the presence of said fad2 nucleic acid.

This method can also comprise a step selected from the group consisting of: obtaining doubled haploid plants containing a fad2 nucleic acid, in vitro cultivation, cloning or asexual reproduction. PCR primers can be used to screen plants, derived from said selfing or crossing, for the presence of said fad2 nucleic acid, said markers being linked to said fad2 allele. In a specific aspect of the invention, the method can be performed using PCR primers OSR144 (SEQ ID No. 7) and OSR145 (SEQ ID No. 8).

The method for detecting the presence or absence of the mutant FAD2 allele in the DNA of Brassica tissue or seeds, can be performed using the mutant FAD2 PCR Identification Protocol (or fad2 PCR Identification protocol).

In a further aspect of the invention, a Brassica plant with a high oleic acid content in its seeds is provided wherein the presence of the fad2 nucleic acid can be detected with at least the PCR primer pair ORS144 (SEQ ID No. 5) and OSR 145 (SEQ ID No. 6).

A further aspect the invention deals with a vegetable oil extracted from seeds of plants comprising the fad2 allele of the invention. Said seeds can be used to produce oilseed rape oil or an oilseed rape seed cake.

According to the invention, a seed cake is defined as the remainder of the seed after crushing the oil out of the seed.

Said plants, according to the invention and comprising the mutant fad2 allele, can be used to produce seed comprising a mutant FAD2 enzyme, oilseed rape oil or an oilseed rape seed cake, or to produce a crop of oilseed rape comprising the mutant FAD2 enzyme.

Unless stated otherwise in the Examples, all recombinant DNA techniques are carried out according to standard protocols as described in Sambrook and Russell (2001) Molecular Cloning: A Laboratory Manual, Third Edition, Cold Spring Harbor Laboratory Press, NY, in Volumes 1 and 2 of Ausubel et at. (1994) Current Protocols in Molecular Biology, Current Protocols, USA and in Volumes I and II of Brown (1998) Molecular Biology LabFax, Second Edition, Academic Press (UK). Standard materials and methods for plant molecular work are described in Plant Molecular Biology Labfax (1993) by R. D. D. Croy, jointly published by BIOS Scientific Publications Ltd (UK) and Blackwell Scientific Publications, UK. Standard materials and methods for polymerase chain reactions can be found in Dieffenbach and Dveksler (1995) PCR Primer A Laboratory Manual, Cold Spring Harbor Laboratory Press, and in McPherson at al. (2000) PCR—Basics: From Background to Bench, First Edition, Springer Verlag, Germany. Standard procedures for AFLP analysis are described in Vos et al. (1995, NAR 23:4407-4414) and in published EP patent application EP 534858.

It should be understood that the preceding is merely a detailed description of particular embodiments of this invention and that numerous changes to the disclosed embodiments can be made in accordance with the disclosure herein without departing from the spirit or scope of the invention. The preceding description, nor the following examples, is meant to limit the scope of the invention. Rather, the scope of the invention is to be determined only by the appended claims and their equivalents.

### Example 1

### Isolation of a Plant with a Mutation in the fad2 Gene

Such a plant can be identified using the TILLING approach. This comprises the following steps: treatment of seed with the mutagen EMS, growing the seeds into M1 plants and self pollination to obtain M2 seeds in order to create a TILLING library; DNA samples from individual M2 seeds are pooled 4-10 fold; FAD2 specific unlabeled primers and identical primers labeled at the 5′ end with the fluorescent dye IRD700 or IRD800 and approximately 1000 bp apart are mixed and used in a PCR amplification; after amplification samples are digested with Cell enzyme (Surveyor Kit) and denatured; fragments are separated using a polyacrylamide gel on a LI-COR² gel analyzer; images can be analysed visually for the presence of cleavage products indicating a potential point mutation in the FAD2 gene.

### Example 2

### Oilseed Rape Line with Elevated Levels of Oleic Acid in the Seed Oil

A winter oilseed rape line (HOWOSR) was identified as comprising a FAD2 mutation and the fatty acid content of its seed oil was analyzed. More particularly, the oleic acid content of its seed oil was determined. This line was grown in the field at two different locations and the fatty acid composition of its seed oil was analysed. For the sake of comparison, the fatty acid composition of the Express variety, grown in the same conditions is indicated. This variety does not comprise any mutation in its FAD2 genes.

The fatty acid composition of the seed oil was analyzed as follow:

The seed samples were dried and weighted. 0.8 g of seeds were put into plastic vials. A steel crushing rod was added to each vial. This vial was then filled with 2 ml methylation solution (10 g sodium methoxide in 500 ml methanol) and 0.8 ml of petroleum ether. The capped vials were shaken for 30 min on an Eberbach shaker. One ml of de-ionized water was added to each vial before recapping and shaking. The vials were centrifugated for 5 min at 3500 rpm.

25-50 μl of the petroleum ether layer from each sample were transferred into Gas Chromatography (GC) autosampler vials. 400 μl 0.4 M phosphate buffer and 800 μl petroleum ether were added to each vial before shaking them.

0.5 to 1 μl of the petroleum ether layer of the samples were injected for analysis in the gas chromatograph.

Print out from the gas chromatograph are analyzed for calculation of each fatty acid content.

TABLE 1
Fatty Acid Composition of High Oleic Acid Mutant HOWOSR
(in percentage of total fatty acid)

| | | Palmitic acid | Stearic acid | Oleic acid | Linoleic | Linolenic |
|---|---|---|---|---|---|---|
| Line/location | | (16:0) | (18:0) | (18:1) | acid (18:2) | acid (18:3) |
| Express | France | 4.86 | 1.60 | 63.77 | 18.58 | 8.83 |
| | Belgium | 5.29 | 1.42 | 58.18 | 21.95 | 10.92 |
| HOWOSR | France | 4.03 | 1.51 | 69.01 | 12.95 | 9.73 |
| | Belgium | 4.36 | 1.30 | 68.24 | 12.65 | 11.11 |

### Example 3

### Sequence of the Mutant FAD2 Gene of HOWOSR

Primers were designed in conserved regions of FAD2 genes from different plant species using alignment of publicly available sequences.

These primers were then used in PCR reactions to amplify differents parts of the FAD2 sequence of the HOWOSR line. The PCR products were then cloned in vectors and sequenced. Alignment of the PCR products indicated that the PCR products comprised two different FAD2 sequences.

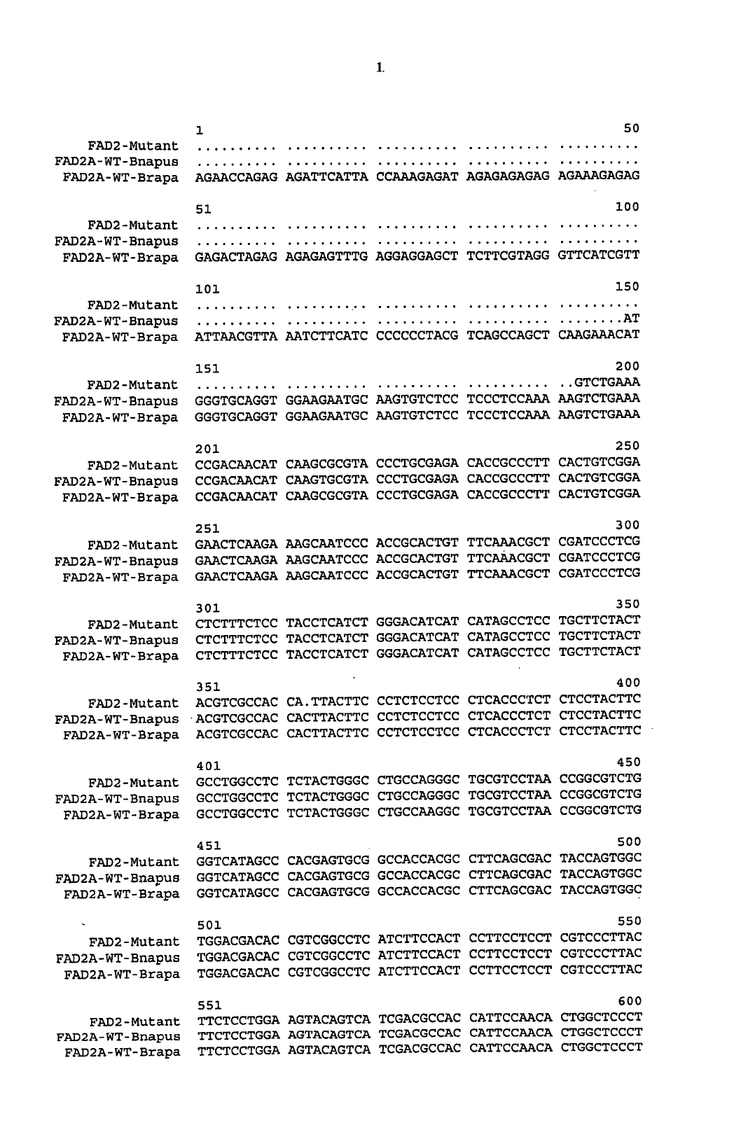

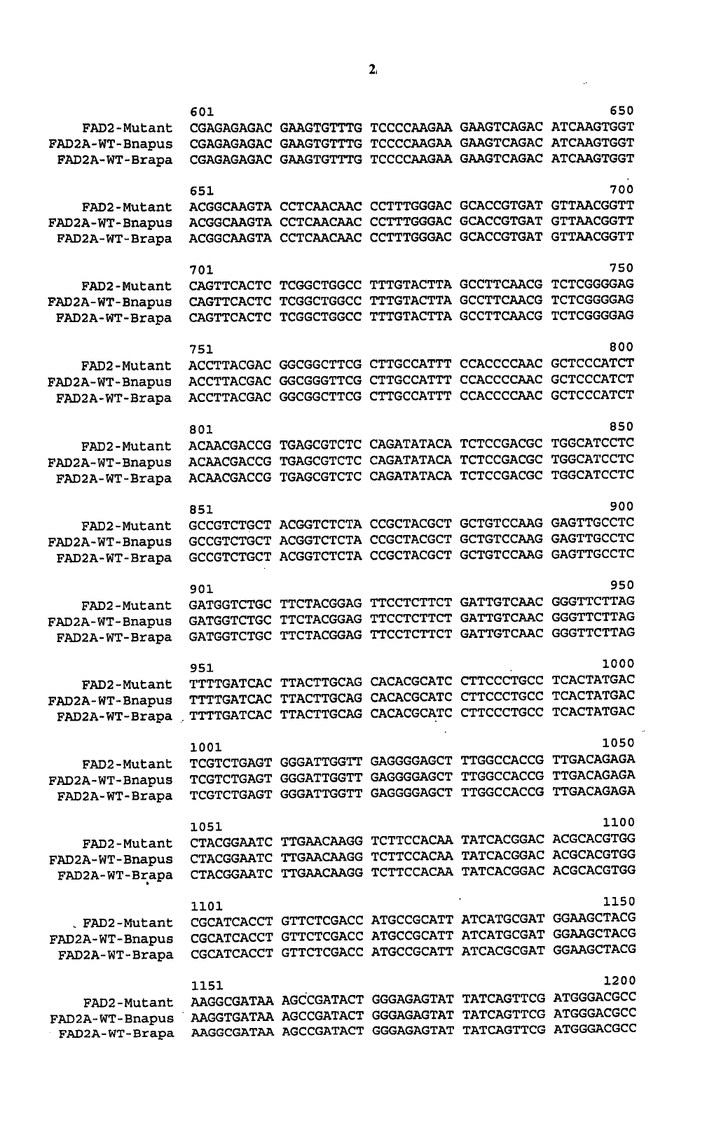

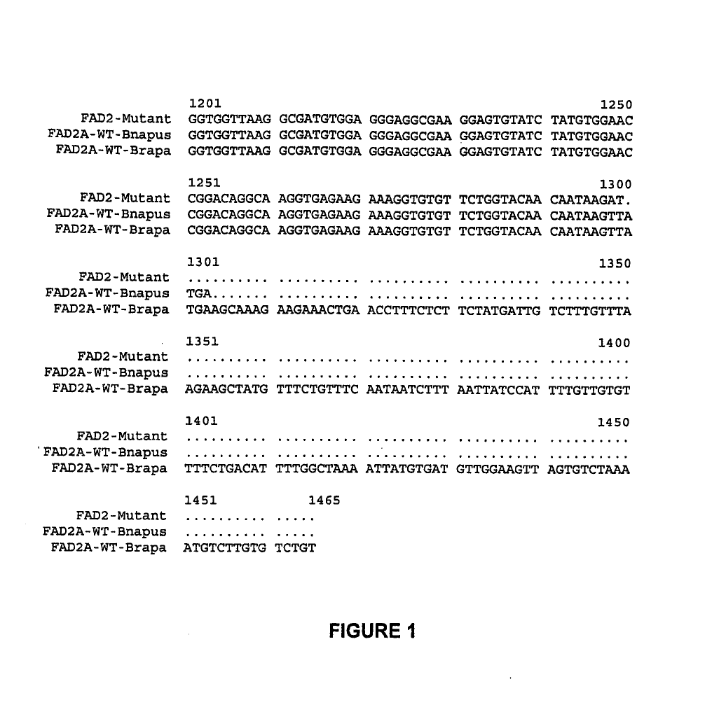

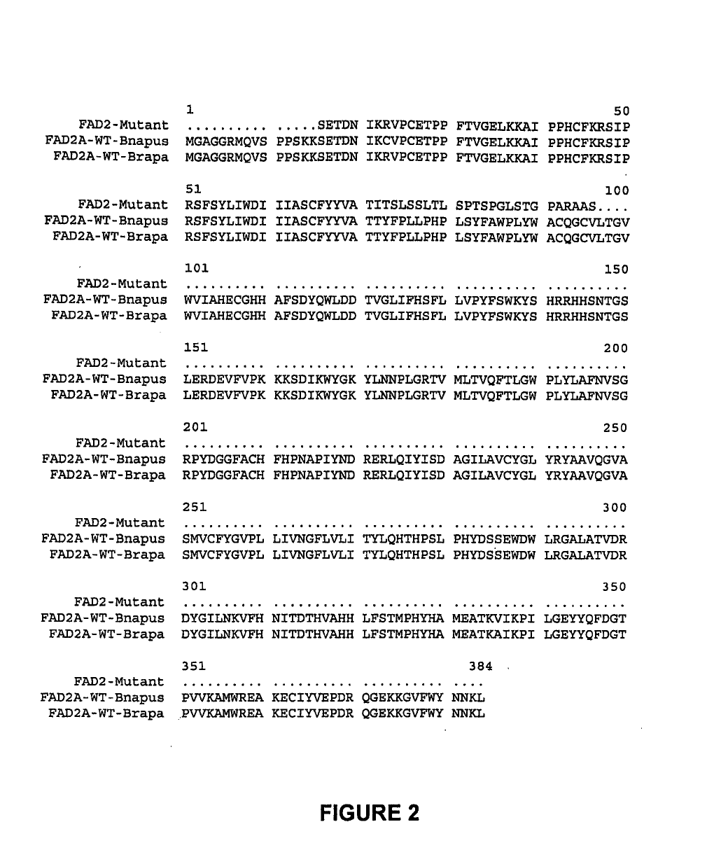

One of them, represented as SEQ ID No. 3, showed a high sequence identity with the sequence of the FAD2 gene from Brassica rapa (SEQ ID NO. 1), indicating a A-genome origin. FIG. 1 presents the alignement of the FAD2 sequence isolated from HOWOSR (SEQ ID No. 3), FAD2 gene from Brassica rapa (SEQ ID NO. 1) and FAD2 gene from Brassica napus (SEQ ID No. 2). This sequence contains a deletion at base 171 of SEQ ID No 3 causing a frameshift mutation that results in nonsense translation and premature stop. The resulting polypeptide, comprising SEQ ID No. 4, is assumed to be completely non-functional. FIG. 2 represents the alignement of the amino acid sequence of the FAD2 protein from B. rapa (SEQ ID No. 6), B. napus (SEQ ID No. 5) and the FAD2 mutant protein of the invention (SEQ ID No. 4).

The other one after alignment with public databases, presents a correlation with the sequence of a FAD2 gene from Brassica oleracea indicating a C-genome origin (SEQ ID No. 2).

### Example 4

### Protocols for the PCR-Based Detection of the Mutant and the Wild-Type Allele of the FAD2 Gene of HOWOSR

A PCR assay distinguishing the mutant FAD2 allele (fad2) of the present invention from the wild-type FAD2 allele along with an analysis of the fatty acid composition of the seed oil, provides a means to simplify segregation and selection analysis of genetic crosses involving plants having the mutant FAD2 allele (fad2).

A PCR protocol was developed to determine the presence or absence of the mutant and the wild-type allele of the FAD2 gene of Brassica napus, the mutant FAD2 PCR identification protocol. The assay is separated in two PCR reactions, one to detect the mutant FAD2 allele and one to detect the wild type FAD2 allele. To detect the mutant allele, only the mutant PCR assay has to be performed. To obtain information about the zygosity status of the plant, both the mutant and the wild type PCR assays have to be performed. Each reaction includes also primers for a native endogenous control gene. One has to attain PCR and thermocycling conditions that amplify equimolar quantities of both the endogenous and target sequence in a known genomic DNA template. A validation test should be performed, including appropriate controls, before attempting to screen unknowns. The present fad2 identification protocol may require minor optimization for various criteria that may differ between laboratories (template DNA preparation, Taq DNA polymerase, quality of the primers, dNTP's, thermocycler types, etc.).

### 1. Primers

Detection of Mutant FAD2 Allele (fad2):

OSR144 (SEQ ID No.7):
5′-ACT.ACg.TCg.CCA.CCA.TTA.C-3′ 19-mer
OSR145 (SEQ ID No.8):
5′-ggA.gCC.AgT.gTT.ggA.ATg.g-3′ 19-mer

The use of this primer pair generates an amplified fragment of about 250 bp

### Detection of Wild-Type FAD2 Allele:

OSR146 (SEQ ID No.9):
5′-CTA.CTA.CgT.CgC.CAC.CAC-3′ 18-mer
OSR147 (SEQ ID No.10):
5′-ACg.CCg.gTT.Agg.ACg.CAg-3′ 18-mer

The use of this primer pair generates an amplified fragment of 101 bp

### Endogenous Primers

OSR001 (SEQ ID No.11):
5′-AAC.gAg.TgT.CAg.CTA.gAC.CAg.C-3′ 22-mer
OSR002 (SEQ ID No.12):
5′-CgC.AgT.TCT.gTg.AAC.ATC.gAC.C-3′ 22-mer

The use of this primer pair generates an amplified fragment of 394 bp

### 2. Components for One 25 μl Reaction

Detection of Mutant FAD2 Allele (fad2):

X μl template DNA (50 ng)

2.5 μl 10×PCR buffer

2.0 μl OSR144 [10 pmol/μl]

2.0 μl OSR145 [10 pmol/μl]

0.2 μl OSR001 [10 pmol/μl]

0.2 μl OSR002 [10 pmol/μl]

0.1 μl Taq DNA polymerase

0.375 μl 10 mM dNTPs

H2O up to 25 μl

### Detection of Wild-Type FAD2 Allele:

X μl template DNA (50 ng)

2.5 μl 10×PCR buffer

1.0 μl OSR146 [10 pmol/μl]

1.0 μl OSR147 [10 pmol/μl]

0.2 μl OSR001 [10 pmol/μl]

0.2 μl OSR002 [10 pmol/μl]

0.1 μl Taq DNA polymerase

0.375 μl 10 mM dNTPs

H2O up to 25 μl

### 3. Thermocycling Profile:

Detection of Mutant FAD2 Allele (fad2):

4 min. at 95° C.

Followed by:

1 min. at 95° C.

1 min. at 57° C.

2 min. at 72° C.

For 5 cycles

Followed by:

30 sec. at 92° C.

30 sec. at 57° C.

1 min. at 72° C.

For 25 cycles

Followed by:

5 minutes at 72° C.

### Detection of Wild-Type FAD2 Allele:

4 min. at 95° C.

Followed by:

1 min. at 95° C.

1 min. at 60° C.

2 min. at 72° C.

For 5 cycles

Followed by:

30 sec. at 92° C.

30 sec. at 60° C.

1 min. at 72° C

For 25 cycles

Followed by:

5 minutes at 72° C.

### Example 5

### Analysis of the Correlation between the Presence of the Mutant FAD2 Allele (fad2) from HOWOSR and the Level of Oleic and Linoleic Acid in Seed Oil

1. HOWOSR was crossed with an elite winter B. napus line (PP0150-0011b) to determine the correlation between the presence of the mutant FAD2 allele (fad2) from HOWOSR in homozygous and heterozygous state and the level of oleic and linoleic acid in the seed oil of the progeny plants in the greenhouse.

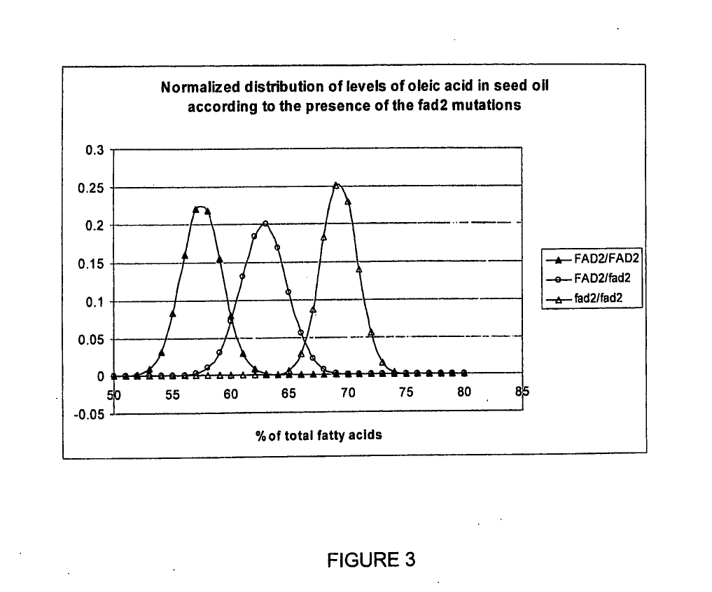

The presence of the fad2 allele from HOWOSR in F2 plants explained the observed differences in oleic acid content of seed oil from the F2 plants. Oleic acid (C18:1) levels raised from about 57.5% in seed oil of plants not comprising the mutant FAD2 allele (indicated as “FAD2/FAD2” in FIG. 2) to about 69.3% in seed oil of plants comprising the mutant FAD2 allele in homozygous state (“fad2/fad2”). Oleic acid levels in seed oil from plants comprising the mutant FAD2 allele in heterozygous state (“FAD2/fad2”) were Intermediate (additive affect) (FIG. 3).

The level of linoleic acid (C18:2) decreased from about 22.0% in seed oil of plants not comprising the mutant FAD2 allele (“FAD2/FAD2”) to about 11.1% in seed oil of plants comprising the mutant FAD2 allele in homozygous state (“fad2/fad2”).

The level of linolenic acid (C18:3) was about 10.8% in seed oil of plants not comprising the mutant FAD2 allele (“FAD2/FAD2”) and about 10.1% in seed oil of plants comprising the mutant FAD2 allele in homozygous state (“fad2/fad2”).

The analysis of the fatty acid content was made according to the protocol described in Example 2.

2. HOWOSR was crossed with an elite winter B. napus line (PP0150-0011b) line to determine the correlation between the presence of the mutant FAD2 allele from HOWOSR in homozygous and heterozygous state and the level of oleic and linoleic acid in the seed oil of the progeny plants in the field.

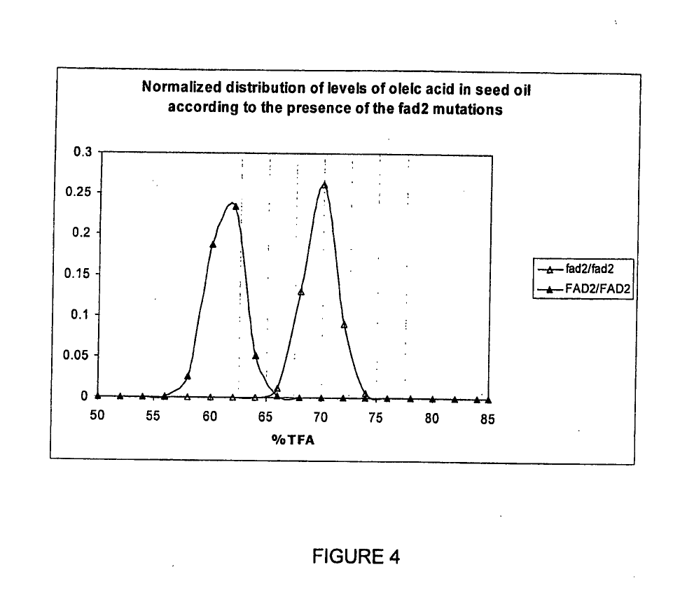

The presence of the mutant FAD2 allele from HOWOSR in doubled haploid (DH) plants derived from the F1 plants explained the observed differences in oleic acid content of seed oil from the DH plants. Oleic acid (18:1) levels raised from about 61.3% In seed oil of plants not comprising the mutant FAD2 allele (indicated as “FAD2/FAD2” in FIG. 2) to 69.8% in seed oil of plants comprising the mutant FAD2 allele in homozygous state (“fad2/fad2”) (FIG. 4).

The level of linoleic acid (C18:2) decreased from about 19.1% in seed oil of plants not comprising the mutant FAD2 allele (“FAD2/FAD2”) to about 11.2% in seed oil of plants comprising the mutant FAD2 allele in homozygous state (“fad2/fad2”).

The level of linolenic acid (C18:3) was about 10.2% in seed oil of plants not comprising the mutant FAD2 allele (“FAD2/FAD2”) and about 10.0% in seed oil of plants comprising the mutant FAD2 allele in homozygous state (“fad2/fad2”).

### Example 6

### Analysis of the Correlation between the Presence of the Mutant FAD2 Allele from HOWOSR and the Mutant FAD3A and FAD3C Alleles from B3119 Stellar and the Level of Oleic, Linoleic, and Linolenic Acid in Seed Oil

HOWOSR was crossed with B3119 Stellar (Spring B. napus variety known as bearing mutations in the FAD3 genes of the A and C genomes, Jourdren et al., 1996, Euphytica, 90: 351-359) to determine the correlation between the presence of the mutant FAD2 allele from HOWOSR and the mutant. FAD3A (fad3a) and FAD3C (fad3c) alleles from B3119 Stellar and the level of oleic, linoleic, and linolenic acid in the seed oil of the progeny plants in the field.

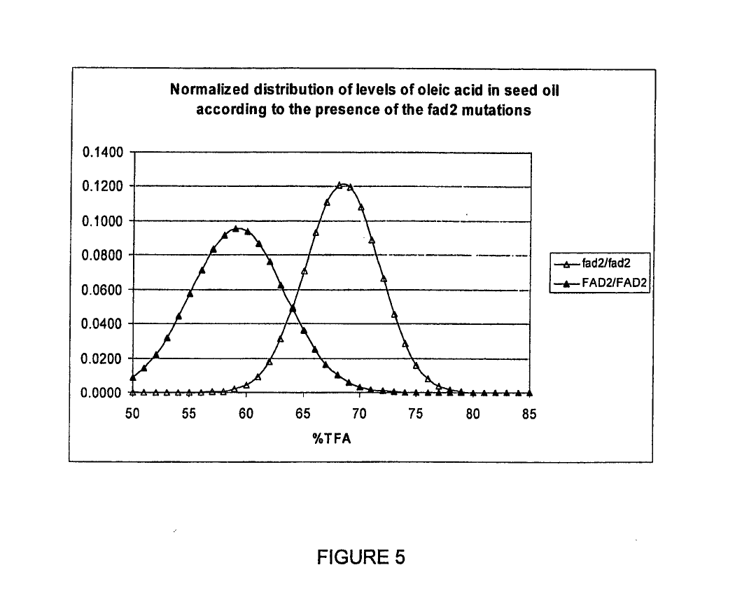

The presence of the mutant FAD2 allele from HOWOSR in doubled haploid (DH) plants derived from the F1 plants, raised the oleic acid (C18:1) levels from about 59.2% in seed oil of plants not comprising the mutant FAD2 allele (indicated as “FAD2/FAD2” in FIG. 5 and Table 2) to about 68.4% in seed oil of plants comprising the mutant FAD2 allele in homozygous state (“fad2/fad2”)(FIG. 4 and Table 2).

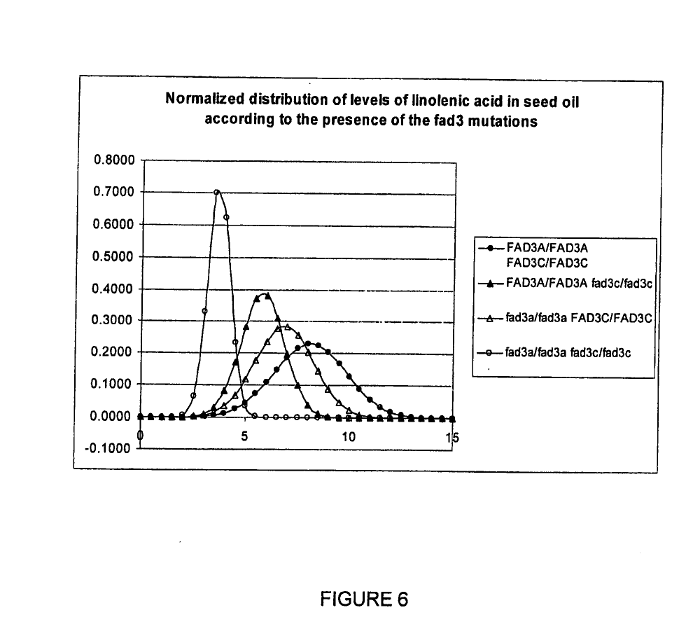

The presence of the mutant FAD3A and FAD3C alleles from B3119 Stellar in doubled haploid (DH) plants derived from the F1 plants, reduced the linolenic acid (C18:3) levels from about 8.4% in seed oil of plants not comprising the mutant FAD3A and FAD3C alleles (indicated as “FAD3A/FAD3A FAD3C/FAD3C” in FIG. 6 and Table 2) to about 3.6% in seed oil of plants comprising the mutant FAD3-A and FAD3-C alleles (“fad3a/fad3a fad3c/fad3c”). Linolenic acid levels in seed oil from plants comprising either the mutant FAD3A allele (“fad3a/fad3a FAD3C/FAD3C”) or the mutant FAD3-C allele (“FAD3A/FAD3A fad3c/fad3c”) were intermediate (additive affect)(FIG. 5 and Table 2).

The average level of linoleic acid (C18:2) decreased from about 20.7% in seed oil of plants not comprising the mutant FAD2 allele from HOWOSR nor the mutant FAD3-A and FAD3-C alleles from B3119 Stellar (indicated as “FAD2/FAD2 FAD3A/FAD3A FAD3C/FAD3C” in Table 2) to about 17.7% in seed oil of plants comprising the mutant FAD2 allele from HOWOSR and the mutant FAD3-A and FAD3-C alleles from B3119 Stellar (indicated as “fad2/fad2 fad3a/fad3a fad3c/fad3c” in Table 2) (Table 2).

TABLE 2

| | | | C18:1_avg | C18:2_avg | C18:3_avg |
|---|---|---|---|---|---|
| fad2/fad2 | fad3a/fad3a | fad3c/fad3c | 68.6% | 17.7% | 3.6% |
| fad2/fad2 | FAD3A/FAD3A | fad3c/fad3c | 69.2% | 14.9% | 5.6% |
| fad2/fad2 | fad3a/fad3a | FAD3C/FAD3C | 66.6% | 16.4% | 6.8% |
| fad2/fad2 | FAD3A/FAD3A | FAD3C/FAD3C | 69.4% | 13.0% | 7.8% |
| FAD2/FAD2 | fad3a/fad3a | fad3c/fad3c | 57.9% | 28.1% | 3.8% |
| FAD2/FAD2 | FAD3A/FAD3A | fad3c/fad3c | 59.8% | 23.7% | 6.1% |
| FAD2/FAD2 | fad3a/fad3a | FAD3C/FAD3C | 58.8% | 23.8% | 6.9% |
| FAD2/FAD2 | FAD3A/FAD3A | FAD3C/FAD3C | 61.0% | 20.7% | 8.4% |

### Example 7

### Transfer of the Mutant FAD2 Allele into other Brassica Elite Lines

The mutant FAD2 allele is transferred into other elite breeding lines by the following method. A plant containing the mutant FAD2 allele (donor plant), is crossed with an elite Brassica line (elite parent/recurrent parent) or variety lacking the mutant FAD2 allele. The following introgression scheme is used (the mutant FAD2 allele is abbreviated to fad2):

Initial cross: fad2/fad2 (donor plant) X FAD2/FAD2 (elite parent)

F1 plant: FAD2/fad2

BC1 cross: FAD2/fad2 X FAD2/FAD2 (recurrent parent)

BC1 plants: 50% FAD2/fad2 and 50% FAD2/FAD2

The 50% FAD2/fad2 are selected using the PCR markers for the mutant FAD2 allele (fad2)

BC2 cross: FAD2/fad2 (BC1 plant) X FAD2/FAD2 (recurrent parent)

BC2 plants: 50% FAD2/fad2 and 50% FAD2/FAD2

The 50% FAD2/fad2 are selected using the PCR markers for the mutant FAD2 allele (fad2)

Backcrossing is repeated until BC6

BC6 plants: 50% FAD2/fad2 and 50% FAD2/FAD2

The 50% FAD2/fad2 are selected using AFLP markers or the PCR marker for the mutant FAD2 allele (fad2)

BC6 S1 cross: FAD2/fad2 X FAD2/fad2

BC6 S1 plants: 25% FAD2/FAD2 and 50% FAD2/fad2 and 25% fad2/fad2

Plants containing fad2 are selected using the PCR markers for the mutant FAD2 allele

Individual BC6 S1 plants that are homozygous for the mutant FAD2 allele (fad2/fad2) are selected using PCR markers for fad2 (select on presence) and PCR markers for FAD2 (select for absence). These plants are then used for seed production.

1. An isolated nucleic acid encoding a FAD2 desaturase, the nucleotide sequence of which comprises a nucleotide deletion.

2. The nucleic acid according to claim 1, wherein the nucleotide sequence comprises SEQ ID No. 3.

3. An isolated FAD2 polypeptide encoded by a nucleic acid, the nucleotide sequence of which comprises a nucleotide deletion, said FAD2 polypeptide being non functional.

4. The polypeptide according to claim 3, comprising the amino acid sequence of SEQ ID No. 4.

5. A plant cell comprising the nucleic acid of claim 1.

6. A plant cell expressing the mutant FAD2 polypeptide according to claim 3.

7. A Brassica plant with a high level of oleic acid in its seed oil, comprising a mutant FAD2 allele.

8. The Brassica plant according to claim 7 with high oleic content in its seed oil, wherein said mutant FAD2 allele comprises a nucleic acid encoding a FAD2 desaturase; the nucleotide sequence of which comprises a nucleotide deletion.

9. The Brassica plant according to claim 7, wherein said mutant FAD2 allele expresses a FAD2 polypeptide encoded by a nucleic acid, the nucleotide sequence of which comprises a nucleotide deletion said FAD2 polypeptide being non functional.

10. The Brassica plant according to claim 7 with high oleic content in its seed oil, wherein said Brassica plant is selected from the group consisting of:
a Brassica plant containing a transgene integrated into its genome,
a Brassica plant that contains a level of aliphatic glucosinolates in dry, defatted seed meal of less than 30 μmol/g,
a Brassica plant the solid component of the seed contains less than 30 micromoles of any one or any mixture of 3-butenyl glucosinolate, 4-pentenyl glucosinolate, 3-hydroxy-3 butenyl glucosinolate, and 2-hydroxy-4-pentenyl glucosinolate per gram of air-dry, oil-free solid,
a Brassica plant that produces an oil containing less than 2% erucic acid of the total fatty acids in the oil,
a Brassica napus plant
a B. napus spring oilseed rape plant,
a B. napus winter oilseed rape plant,
progeny of a Brassica plant containing said mutant FAD2 nucleic acid, wherein said progeny results from crosses between Brassica plants containing said mutant FAD2 nucleic acid and a Brassica variety with low linolenic acid content in its seeds or a herbicide resistant Brassica variety.

11. The Brassica plant according to claim 7 with high oleic content in its seed oils, wherein the presence of the mutant FAD2 allele can be detected with at least the PCR primer pair OSR144 (SEQ ID No. 7) and OSR145 (SEQ ID No. 8).

12. A seed of a plant according to claim 7 comprising said mutant FAD2 allele.

13. The seed according to claim 12, wherein said seed is a hybrid seed.

14. Hybrid Brassica seeds, comprising the mutant FAD2 allele, which develop into plants, wherein the solid component of the seeds of said plants contains less than 30 micromoles of any one or any mixture of 3-butenyl glucosinolate, 4-pentenyl glucosinolate, 3-hydroxy-3 butenyl glucosinolate, and 2-hydroxy-4-pentenyl glucosinolate per gram of air-dry, oil-free solid component, and which produces an seed oil containing less than 2% erucic acid of the total fatty acids in the oil.

15. The hybrid Brassica seeds of claim 14, wherein said mutant FAD2 allele comprises a nucleic acid encoding a FAD2 desaturase, the nucleotide sequence of which comprises a nucleotide deletion.

16. The hybrid Brassica seeds of claim 14, wherein the mutant FAD2 allele expresses a FAD2 polypeptide encoded by a nucleic acid, the nucleotide sequence of which comprises a nucleotide deletion, said polypeptide being non functional.

17. A vegetable oil extracted from seeds according to claim 12.

18. Plants derived from the hybrid seeds according to claim 13.

19. A method for transferring the mutant FAD2 allele from one Brassica plant into another Brassica plant, comprising crossing the Brassica plant according to claim 7 with another Brassica plant, collecting F1 seeds from said cross, selfing or crossing the F1 plants derived from said F1 seeds for one or more generations and screening plants derived from said selfing or crossing for the presence of said mutant FAD2 allele.

20. The method of claim 19, which also comprises the step selected from the group consisting of: obtaining doubled haploid plants containing said mutant FAD2 allele fad2 nucleic acid, in vitro cultivation, cloning or asexual reproduction.

21. The method according to claim 19, wherein said screening is done using a PCR primer pair specific for said mutant FAD2 nucleic acid.

22. The method according to claim 21 wherein said PCR primer pair comprises PCR primer OSR144 (SEQ ID No. 7) and PCR primer OSR145 (SEQ ID No. 8).

23. The method according to claim 19, wherein said screening is done according to the mutant FAD2 PCR Identification Protocol.

24. A method for detecting the presence or absence of the mutant FAD2 allele in the DNA of Brassica tissue or seeds, comprising performing the mutant FAD2 PCR Identification Protocol.

25. A kit for the detection of the mutant FAD2 allele in Brassica DNA samples, wherein said kit comprises one or more PCR primer pairs, which are able to amplify a DNA marker linked to the mutant FAD2 allele.

26. The kit according to claim 25, wherein said PCR primer pairs are selected from primer pairs OSR144 (SEQ ID No. 7)-OSR145 (SEQ ID No. 8), OSR146 (SEQ ID No. 9)-OSR147 (SEQ ID No. 10) and ORS001 (SEQ ID No. 11)-OSR002 (SEQ ID No. 12).

27. The kit according to claim 26, wherein said primer pairs are able to amplify a DNA fragment of about 250 bp, about 101 bp, and about 394 bp, respectively.

28. The kit according to claim 25, further comprising seeds or tissue, wherein DNA extracted from said seeds or tissue can be used as a positive or negative control.

29. A PCR marker primer for Brassica, selected from the group consisting of OSR144 (SEQ ID No. 7) and OSR145 (SEQ ID No. 8).

30. Use of any one of PCR markers primers for monitoring the introgression of mutant FAD2 allele in Brassica oilseed plants or for PCR analysis of Brassica oilseed plants.

31. Use of claim 30 wherein said PCR markers primers are OSR144 (SEQ ID No. 7) and OSR145 (SEQ ID No. 8) for monitoring the introgression of mutant FAD2 allele in Brassica.

32. Use of the plant of claim 7 to produce oilseed rape oil or an oilseed rape seed cake.

33. Use of the seeds of claim 12 to produce oilseed rape oil or an oilseed rape seed cake.

34. Use of the plant of claim 7 to produce seed comprising a mutant FAD2 enzyme.

35. Use of the plant of claim 7 to produce a crop of oilseed rape, comprising mutant FAD2 enzyme.

36. The Brassica plant according to claim 7 with high oleic content in its seed oil, wherein said mutant FAD2 allele comprises a nucleic acid encoding a FAD2 desaturase, the nucleotide sequence of which comprises SEQ ID NO: 3.

37. The Brassica plant according to claim 7, wherein said mutant FAD2 allele expresses a FAD2 polypeptide comprising the amino acid sequence of SEQ ID NO: 4.

38. The hybrid Brassica seeds of claim 14, wherein said mutant FAD2 allele comprises a nucleic acid encoding a FAD2 desaturase, the nucleotide sequence of which comprises SEQ ID NO: 3.

39. The hybrid Brassica seeds of claim 14, wherein the mutant FAD2 allele expresses a FAD2 polypeptide comprising the amino acid sequence of SEQ ID NO: 4.


SEQUENCE LISTING (12 sequences)

SEQ ID NO: 1 - DNA, 1465 bp, Brassica rapa
agaaccagagagattcattaccaaagagatagagagagagagaaagagaggagactagagagagagtttgaggaggagcttcttcgtagggttcatcgttattaacgttaaatcttcatccccccctacgtcagccagctcaagaaacatgggtgcaggtggaagaatgcaagtgtctcctccctccaaaaagtctgaaaccgacaacatcaagcgcgtaccctgcgagacaccgcccttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccaaggctgcgtcctaaccggcgtctgggtcatagcccacgagtgcggccaccacgccttcagcgactaccagtggctggacgacaccgtcggcctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggcctttgtacttagccttcaacgtctcggggagaccttacgacggcggcttcgcttgccatttccaccccaacgctcccatctacaacgaccgtgagcgtctccagatatacatctccgacgctggcatcctcgccgtctgctacggtctctaccgctacgctgctgtccaaggagttgcctcgatggtctgcttctacggagttcctcttctgattgtcaacgggttcttagttttgatcacttacttgcagcacacgcatccttccctgcctcactatgactcgtctgagtgggattggttgaggggagctttggccaccgttgacagagactacggaatcttgaacaaggtcttccacaatatcacggacacgcacgtggcgcatcacctgttctcgaccatgccgcattatcacgcgatggaagctacgaaggcgataaagccgatactgggagagtattatcagttcgatgggacgccggtggttaaggcgatgtggagggaggcgaaggagtgtatctatgtggaaccggacaggcaaggtgagaagaaaggtgtgttctggtacaacaataagttatgaagcaaagaagaaactgaacctttctcttctatgattgtctttgtttaagaagctatgtttctgtttcaataatctttaattatccattttgttgtgttttctgacattttggctaaaattatgtgatgttggaagttagtgtctaaaatgtcttgtgtctgt

SEQ ID NO: 2 - DNA, 1155 bp, Brassica napus
atgggtgcaggtggaagaatgcaagtgtctcctccctccaaaaagtctgaaaccgacaacatcaagtgcgtaccctgcgagacaccgcccttcactgtcggagaactcaagaaagcaatcccaccgcactgtttcaaacgctcgatccctcgctctttctcctacctcatctgggacatcatcatagcctcctgcttctactacgtcgccaccacttacttccctctcctccctcaccctctctcctacttcgcctggcctctctactgggcctgccagggctgcgtcctaaccggcgtctgggtcatagcccacgagtgcggccaccacgccttcagcgactaccagtggctggacgacaccgtcggcctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggcctttgtacttagccttcaacgtctcggggagaccttacgacggcgggttcgcttgccatttccaccccaacgctcccatctacaacgaccgtgagcgtctccagatatacatctccgacgctggcatcctcgccgtctgctacggtctctaccgctacgctgctgtccaaggagttgcctcgatggtctgcttctacggagttcctcttctgattgtcaacgggttcttagttttgatcacttacttgcagcacacgcatccttccctgcctcactatgactcgtctgagtgggattggttgaggggagctttggccaccgttgacagagactacggaatcttgaacaaggtcttccacaatatcacggacacgcacgtggcgcatcacctgttctcgaccatgccgcattatcatgcgatggaagctacgaaggtgataaagccgatactgggagagtattatcagttcgatgggacgccggtggttaaggcgatgtggagggaggcgaaggagtgtatctatgtggaaccggacaggcaaggtgagaagaaaggtgtgttctggtacaacaataagttatga

SEQ ID NO: 3 - DNA, 1106 bp, Brassica napus
gtctgaaaccgacaacatcaagcgcgtaccctgcgagacaccgcccttcactgtcggagaactcaagaaagcaatcccaccgcactgtttcaaacgctcgatccctcgctctttctcctacctcatctgggacatcatcatagcctcctgcttctactacgtcgccaccattacttccctctcctccctcaccctctctcctacttcgcctggcctctctactgggcctgccagggctgcgtcctaaccggcgtctgggtcatagcccacgagtgcggccaccacgccttcagcgactaccagtggctggacgacaccgtcggcctcatcttccactccttcctcctcgtcccttacttctcctggaagtacagtcatcgacgccaccattccaacactggctccctcgagagagacgaagtgtttgtccccaagaagaagtcagacatcaagtggtacggcaagtacctcaacaaccctttgggacgcaccgtgatgttaacggttcagttcactctcggctggcctttgtacttagccttcaacgtctcggggagaccttacgacggcggcttcgcttgccatttccaccccaacgctcccatctacaacgaccgtgagcgtctccagatatacatctccgacgctggcatcctcgccgtctgctacggtctctaccgctacgctgctgtccaaggagttgcctcgatggtctgcttctacggagttcctcttctgattgtcaacgggttcttagttttgatcacttacttgcagcacacgcatccttccctgcctcactatgactcgtctgagtgggattggttgaggggagctttggccaccgttgacagagactacggaatcttgaacaaggtcttccacaatatcacggacacgcacgtggcgcatcacctgttctcgaccatgccgcattatcatgcgatggaagctacgaaggcgataaagccgatactgggagagtattatcagttcgatgggacgccggtggttaaggcgatgtggagggaggcgaaggagtgtatctatgtggaaccggacaggcaaggtgagaagaaaggtgtgttctggtacaacaataagat

SEQ ID NO: 4 - protein, 81 aa, Brassica napus
SETDNIKRVPCETPPFTVGELKKAIPPHCFKRSIPRSFSYLIWDIIIASCFYYVATITSLSSLTLSPTSPGLSTGPARAAS

SEQ ID NO: 5 - protein, 384 aa, Brassica napus
MGAGGRMQVSPPSKKSETDNIKCVPCETPPFTVGELKKAIPPHCFKRSIPRSFSYLIWDIIIASCFYYVATTYFPLLPHPLSYFAWPLYWACQGCVLTGVWVIAHECGHHAFSDYQWLDDTVGLIFHSFLLVPYFSWKYSHRRHHSNTGSLERDEVFVPKKKSDIKWYGKYLNNPLGRTVMLTVQFTLGWPLYLAFNVSGRPYDGGFACHFHPNAPIYNDRERLQIYISDAGILAVCYGLYRYAAVQGVASMVCFYGVPLLIVNGFLVLITYLQHTHPSLPHYDSSEWDWLRGALATVDRDYGILNKVFHNITDTHVAHHLFSTMPHYHAMEATKVIKPILGEYYQFDGTPVVKAMWREAKECIYVEPDRQGEKKGVFWYNNKL

SEQ ID NO: 6 - protein, 384 aa, Brassica rapa
MGAGGRMQVSPPSKKSETDNIKRVPCETPPFTVGELKKAIPPHCFKRSIPRSFSYLIWDIIIASCFYYVATTYFPLLPHPLSYFAWPLYWACQGCVLTGVWVIAHECGHHAFSDYQWLDDTVGLIFHSFLLVPYFSWKYSHRRHHSNTGSLERDEVFVPKKKSDIKWYGKYLNNPLGRTVMLTVQFTLGWPLYLAFNVSGRPYDGGFACHFHPNAPIYNDRERLQIYISDAGILAVCYGLYRYAAVQGVASMVCFYGVPLLIVNGFLVLITYLQHTHPSLPHYDSSEWDWLRGALATVDRDYGILNKVFHNITDTHVAHHLFSTMPHYHAMEATKAIKPILGEYYQFDGTPVVKAMWREAKECIYVEPDRQGEKKGVFWYNNKL

SEQ ID NO: 7 - DNA, 19 bp, Artificial
  PCR primer
actacgtcgccaccattac

SEQ ID NO: 8 - DNA, 19 bp, Artificial
  PCR primer
ggagccagtgttggaatgg

SEQ ID NO: 9 - DNA, 18 bp, Artificial
  PCR primer
ctactacgtcgccaccac

SEQ ID NO: 10 - DNA, 18 bp, Artificial
  PCR primer
acgccggttaggacgcag

SEQ ID NO: 11 - DNA, 22 bp, Artificial
  PCR primer
aacgagtgtcagctagaccagc

SEQ ID NO: 12 - DNA, 22 bp, Artificial
  PCR primer
cgcagttctgtgaacatcgacc